(19) United States
(12) Patent Application Publication
(10) Pub. No.: US 2012/0253014 A1
(43) Pub. Date: Oct. 4, 2012

(54) PREVENTIVE AGENT FOR ATOPIC DERMATITIS

(75) Inventors: Hiroaki Higuchi, Koshigaya-shi (JP); Atsushi Narise, Kawasaki-shi (JP); Kenji Osawa, Saitama-shi (JP); Katsumasa Shimizu, Tachikawa-shi (JP); Masanori Ito, Misato-shi (JP)

(73) Assignee: Lotte Company, Ltd., Tokyo (JP)

(21) Appl. No.: 13/514,975

(22) PCT Filed: Dec. 7, 2010
(86) PCT No.: PCT/JP2010/007099

§ 371 Date: Jun. 8, 2012

(30) Foreign Application Priority Data
Dec. 10, 2009 (JP) 2009-280606

(51) Int. Cl. C07K 14/78

(52) U.S. Cl. 530/356

(57) ABSTRACT

An object of the present invention is to provide a preventive agent for atopic dermatitis and food products containing the agent. The present inventors conducted extensive studies and, as the result, found that atopic dermatitis can be prevented by orally ingesting collagen. The present invention provides a preventive agent for atopic dermatitis comprising collagen and food products containing the preventive agent for atopic dermatitis.

### TECHNICAL FIELD

The present invention relates to a preventive agent for atopic dermatitis and a food product containing the agent.

### BACKGROUND ART

Atopic dermatitis is a disorder with pruritus eczema, and is a chronic disease which repeats remission and deterioration.

Examples of the primary pathological changes caused by atopic dermatitis include skin erythema or papule, cracks behind the ears, dry skin, keratosis pilaris associated with pityriasisform desquamation and scratch marks on the affected skin. According to the recent research, the prevalence rates of symptoms caused by atopic dermatitis are 12.8% in infants aged 4 months, 9.8% in infants aged 1.5 years, 13.2% in infants aged 3 years, 11.8% in Grade 1 pupils, 10.6% in Grade 6 pupils and 8.2% in year 1 university students, and is as high as one every ten young children. Primary causes and aggravating factors are food, perspiration, environmental factors, bacteria and fungi, contact antigens, stress, etc., and therapeutic and further preventive treatment of this disorder are demanded.

The therapeutic treatment of atopic dermatitis is carried out by 1) detection and counter-measurement of causes and aggravating factors; 2) skin care; and 3) drug therapy. When the symptoms are not alleviated by 1) and 2), the drug therapy is conducted. The most commonly used drug is steroid for external use, which is available in a wide variety. As a non-steroidal drug for external use, on the other hand, Protopic, an immunosuppressive drug, has recently been proved to be useful. Alternatively, oral medicines such as antihistamines, antiallergic drugs, etc., are used, and a steroidal drug for oral administration may temporarily be used for patients with the most severe symptoms. However, steroid, when used as a drug for external use, may cause adverse effects such as atrophy of the skin, vasodilation, folliculitis, etc., and the guideline prepared by Health Labour Sciences Research Group instructs to avoid using a steroid for external use on the face. Many patients also feel uneasy about adverse effects caused by steroids and show rejection. Protopic is a relatively new drug approved in November, 1999, on the other hand, and its use has been approved only in a low concentration for young children and not approved even in a low concentration for children under 2 years of age. Oral medicines such as antihistamines, antiallergic drugs, etc., may cause adverse effects such as drowsiness, fatigue, or difficulty in coughing up of sputum associated with anticholinergic action.

The prevention of atopic dermatitis before the onset, however, has not been much reported so far. However, the number of atopic dermatitis patients is increasing and the prevention against this disorder is desired. In the light of prevention, it is important that safety be assured and adverse effects be eliminated. For this reason, materials used for the prevention are desirably derived from natural products, or better yet, food materials.

Collagen is the main protein component composing the connective tissues in animals, is the raw material for a gelatin and glue, and has been used as a food material for many years. Collagen is also ingested from a meat stew, etc., in everyday life and its safety is widely acknowledged. Collagen is defined as the protein having the collagen triple helical structure and 30 or more types in total have been reported which are respectively termed Type I, Type II, and so on. Type I collagen is the primary component of the derma, ligaments, tendons, bones, and the like; and Type II collagen is the primary component of articular cartilages. Further, Type IV collagen is mainly contained in the basal membrane, which is the undercoat of all of the epithelial tissues. Type I collagen is the most abundant collagen in the body.

It is suggested that application or oral administration of marine collagen controls atopic dermatitis after the onset in atopic dermatitis mouse models (NPL 1). However, the prevention of atopic dermatitis before the onset is not mentioned.

### CITATION LIST

### Non Patent Literature

NPL 1: Northern Advancement Center for Science & Technology, the 2005 (adopted in the fiscal year 2004) Research and development grant project report, p. 161-174

### SUMMARY OF INVENTION

### Technical Problem

An object of the present invention is to provide a preventive agent for atopic dermatitis and a food product containing the agent.

### Solution to Problem

The present inventors conducted extensive studies and, as the result, found that atopic dermatitis can be prevented by orally ingesting collagen, whereby the present invention was accomplished. The present invention provides a preventive agent for atopic dermatitis comprising collagen and a food product containing the preventive agent for atopic dermatitis.

### Advantageous Effects of Invention

Collagen accounts for a large proportion of the total protein in vivo of the mammals, and can be obtained at a low price. Collagen is also the raw material for a gelatin and glue, has been used as a food material for many years and further ingested from a meat stew, etc., in everyday life, whereby its safety is widely acknowledged.

### BRIEF DESCRIPTION OF DRAWINGS

FIG. 1 is a graph showing changes in clinical symptoms score in each group.

FIG. 2 is a graph showing the frequency of scratching in each group.

FIG. 3 is a graph showing the duration of scratching in each group.

FIG. 4 is a graph showing the total IgE levels in blood before and after starting the test in each group.

FIG. 5 is a graph showing TEWL (transepidermal water loss) changes in each group.

FIG. 6 is a graph showing body weight changes in each group.

FIG. 7 is a graph showing the results of macroscopical findings in each group.

FIG. 8 is a graph showing the eosinophil counts and mast cell counts of the head and dorsal skin tissues in each group.

### DESCRIPTION OF EMBODIMENTS

Hereinbelow, the preventive agent for atopic dermatitis comprising collagen of the present invention and a food product containing the agent are described. The collagen of the present invention can be of any origin, and usable are those derived from mammals such as cow, pig, etc., birds such as chicken, ostrich, etc., fishes such as sharks, etc. Those derived from livestock such as cow, pig, chicken, etc., are easily obtained in a large amount, hence particularly preferable. The type of collagen is not limited and any type can be used, or a plurality of collagen types may be used in mixture. Further, collagen may be collagen per se, or gelatin, or furthermore collagen peptide. The gelatin used herein refers to the collagen pre-treated with an acid or alkali and solubilized by heat hydrolysis. The collagen peptide used herein refers to a low molecular collagen obtained by hydrolyzing collagen with an acid, alkali or enzyme. For example, a collagen hydrolyzate can be obtained by immersing skins and joints of animals such as pig, cow and chicken or scales and skins of fish in an acid or alkali solution to extract gelatin and treating the extracted gelatin with an enzyme or acid.

The preventive agent for atopic dermatitis of the present invention is for oral administration, but the dosage form is not limited and can be administered in the form of, for example, tablets, capsules, drinks, etc. The preventive agent for atopic dermatitis of the present invention may further be administered as contained in foods or drinks, and, in that case, foods and drinks in which the agent is contained are not limited. For example, fresh food; animal food products such as meats and fish; plant food products such as grains and vegetables; dairy products; bread; processed food products such as instant food products; non-essential grocery items such as snacks; materials for cooking seasonings such as sweeteners, seasonings; health food products; food products for specific dietary use; beverages such as water, carbonated drinks, alcohol beverages, teas; food processing materials, food additives, etc.

### EXAMPLES

Hereinafter, the present invention is described in reference to the examples, but is not limited thereto.

### Example 1

Verification of the preventive effect of collagen ingestion on dermatitis in NC/Nga Tnd mouse

### Experiment

Whether or not the ingestion of collagen was effective to alleviate symptoms of allergic dermatitis was studied by animal tests. More specifically, NC/Nga Tnd mice as a spontaneously atopic dermatitis mouse model were used for the test.

### Feed

Collagen-containing feed and control feed were used as feed. The collagen-containing feed contains 0.20% of collagen peptide added to the control feed. The collagen-containing feed is adjusted so as to provide a collagen intake of 200 mg/Kg a day. The Jellice Co., Ltd. pig collagen peptide was used as the collagen in the collagen-containing feed. The pig collagen peptide was obtained by immersing the pig skin in an acid or alkali solution to extract gelatin followed by extraction to obtain gelatin, which was and further degraded enzymatically. The present collagen peptide is mainly derived from pig Type I collagen.

### Experiment Items and Details

Male and female NC/Nga Tnd mice, five weeks of age, were divided into two groups, each group containing seven mice. These groups were used as a collagen administration group and a control feed administration group, respectively. After one-week preliminary breeding, the mice of both groups had a six-week free access to the collagen-containing feed and the control feed, respectively, and water. No mice had dermatitis at the time of starting feeding. During the feeding period and before and after the period of each feeding, the mice were assessed for the following seven items. The timings of testing each item are shown in the parenthesis. (1) Assessment of clinical symptom score (twice/week during the feeding period); (2) measurement of the frequency and duration of scratching (before and after the feeding period); (3) assay of the total IgE level in blood (before and after the feeding period); (4) measurement of transepidermal water loss (TEWL) (once/2 weeks during the feeding period); (5) body weight measurement (once/2 weeks during the feeding period); (6) macroscopic observation of skin lesion (at the time of completing the feeding period) and (7) histological examination (after the time of completing the feeding period).

Test Method for each Assessment Item

### (1) Clinical Symptoms Score Assessment

Five items of “pruritus symptom”, “erythema/bleeding”, “edema”, “abrasion/sore” and “desquamation/dry” were categorized into four stages and assessed as “0: none”, “1: mild”, “2: moderate” and “3: severe” on the day before the start of test diet feeding and twice weekly from the day of the first feeding until the day following the last feeding, and the total score of each item is shown. The assessment and feeding were performed by different persons throughout the test period, and the assessment was conducted so that the assessor could not distinguish which group the animals belonged to.

### (2) Measurement of the Frequency and Duration of Scratching

For the purpose of acclimating to the measurement environment, the mice were acclimated to a scratch analyzing system (SCLABA (registered trademark)—Real, Noveltec) for 30 minutes once a day for 2 days from 3 days prior to the start of test feeding. The measurements of the frequency and duration of scratching before the start of feeding were conducted by photographing and recording for 30 minutes after acclimation of the mice for 30 minutes on the day before the start of feeding. On the day following the completion of the feeding period, the mice were acclimated in the same manner followed by photographing and recording the frequency and duration of scratching for 30 minutes. The photographing was performed the period between 12:00 and 18:00 with the date of photographing and ID number recorded.

### (3) Assay of the Total IgE Level in Blood

About 1 mL of blood was collected from the tail vein under ether anesthesia using a heparin-treated syringe before the start of feeding, and from the ventral aorta on the day following the completion of the feeding period and after the photographing and recording the frequency and duration of scratching. The collected blood was centrifuged (4° C.) to separate and cryopreserve (−20° C.) the plasma. The IgE concentration was assayed using the preserved plasma. The IgE assay was carried out by the sandwich ELISA method using anti-mouse IgE antigens (YAMASA, ME-01-DE and ME-02-B) capable of recognizing two different epitopes.

### (4) TEWL Measurement

The measurement was performed twice prior to the start of feeding and after the completion of feeding period, and also once during 2 weeks therebetween. The mouse dorsal part was shaved on the day before the measurement and TEWL of the dorsal part was measured using a multi probe adaptor (CK electronic GmbH). The measurement was carried out three times each time and the average value was defined as TEWL.

### (5) Measurement of Body Weight

Body weight was measured every two weeks from the day before the start of feeding. An electronic balance (HANSEN & CO., LTD., HL-320) was used for the measurement.

### (6) Macroscopical Observation of Skin Lesion

The head, dorsal part and face of mice in each group were photographed at the time of completion of the feeding period.

### (7) Histological Examination

After completion of the feeding period, the dorsal skin was collected, fixed in 10% buffered formalin and paraffin-embedded to prepare thin slices of specimen. The tissue specimens were stained with Congo red dye and toluidine blue, and the number of eosinophils (the specimen stained with Congo red dye) and for the number of mast cells (the specimen stained with toluidine blue) were counted respectively under a microscope in a high power field (400×). The average value of four fields of view per specimen is considered as the individual data, which was totaled by the group.

### Experiment Period

January 17, 2007 to May 25, 2007

### Results

### (1) Clinical Symptom Score Assessment

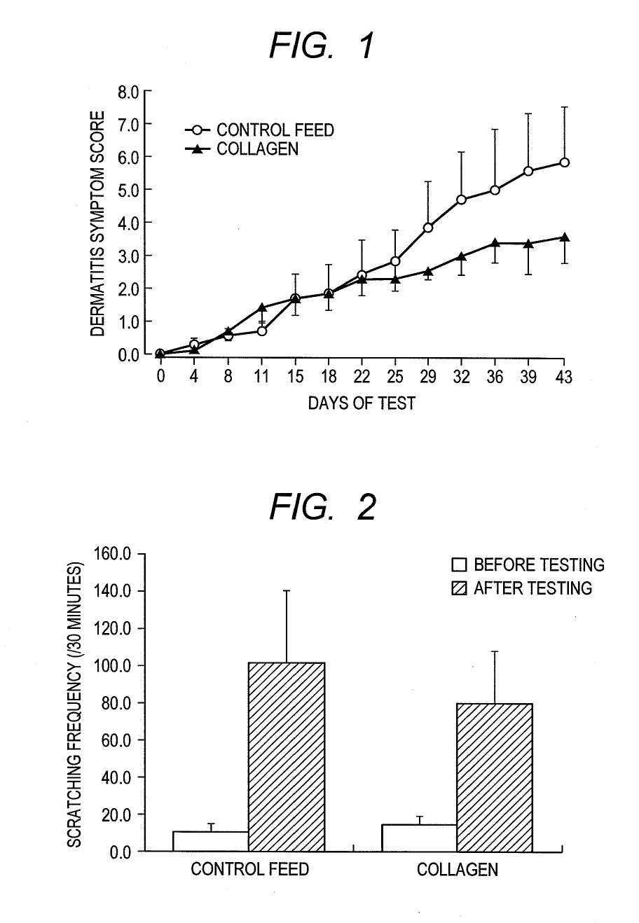

Table 1 and FIG. 1 show the results. FIG. 1 shows the average value±standard error of the clinical symptom score in each group represented by o (control feed group) and ▴ (collagen administration group), respectively.

TABLE 1
Average value and standard error of clinical symptom score in each group

| | | Day measured (Days of test) | | | | | | | | | | | | |
|---|---|---|---|---|---|---|---|---|---|---|---|---|---|---|
| | | 0 | 4 | 8 | 11 | 15 | 18 | 22 | 25 | 29 | 32 | 36 | 39 | 43 |
| Average | Control feed | 0.0 | 0.3 | 0.6 | 0.7 | 1.7 | 1.9 | 2.4 | 2.9 | 3.9 | 4.7 | 5.0 | 5.6 | 5.9 |
| | Collagen | 0.0 | 0.1 | 0.7 | 1.4 | 1.7 | 1.9 | 2.3 | 2.3 | 2.6 | 3.0 | 3.4 | 3.4 | 3.6 |
| SE | Control feed | 0.0 | 0.2 | 0.2 | 0.3 | 0.8 | 0.9 | 1.0 | 0.9 | 1.4 | 1.4 | 1.9 | 1.8 | 1.7 |
| | Collagen | 0.0 | 0.1 | 0.3 | 0.5 | 0.5 | 0.5 | 0.5 | 0.4 | 0.3 | 0.6 | 0.7 | 0.9 | 0.8 |

In both groups, the clinical symptom scores at the time of starting feeding were 0 (undeveloped). The control feed administration group had an elevated clinical symptom score over time starting three days after the start of feeding, and the clinical symptom score was 5.9±1.7 at the completion of the feeding period. The collagen administration group had an increasing dermatitis score in the substantially same manner as in the control feed administration group up to day 25 from the start of feeding, but the dermatitis symptoms were not significantly aggravated on day 25 and after from the start of feeding. The clinical symptom score after completion of the feeding period (day 43) was maintained in the state as mild as 3.6±0.8. The collagen administration group, from day 25 on and after the start of feeding till the completion of the feeding period, tended to have lower clinical symptom scores than the control feed group, although there was no statistically significant difference.

### (2) Measurement of the Frequency and Duration of Scratching

FIG. 2 shows the frequency of scratching behavior before and after the feeding period (for 30 minutes) in each group. The data before the start of feeding (day 0) and after the completion of the feeding period (day 43) were shown as the average value±standard error (7 mice per group).

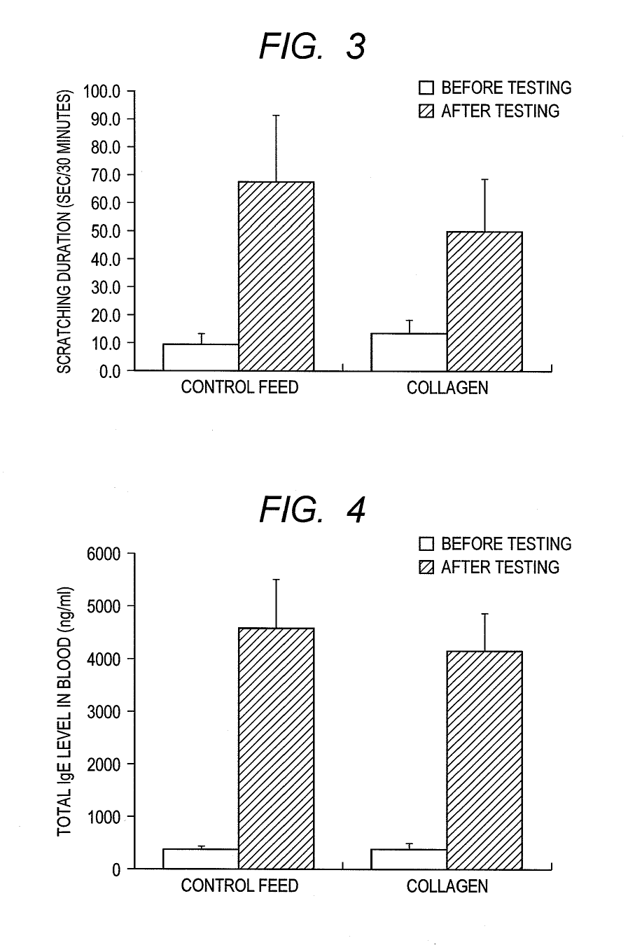

FIG. 3 shows the duration of scratching behavior (sec/30 minutes) before and after the feeding period in each group. The data before the start of feeding (day 0) and after the completion of the feeding period (day 43) are shown as the average value±standard error (7 mice per group).

The scratching frequency and the scratching duration (sec) before the start of feeding (test day 0) in both groups were 10 times and about 10 seconds, respectively, per 30-minute photographing time. Both groups showed increasing tendencies in the scratching frequency and the scratching duration after the completion of the feeding period (day 43) in comparison with before starting feeding, but the collagen administration group had lower scratching frequency and scratching duration than the control feed administration group, although there was no significant difference.

### (3) Assay of the Total IgE Level in Blood

FIG. 4 shows the total IgE levels in blood before and after the feeding period in each group. The data taken before the start of feeding (day 0) and after the completion of the feeding period (day 43) were shown as the average value ±standard error (7 mice per group).

The total IgE level in blood (ng/ml) before the start of feeding (day 0) was about 500 ng/ml, and there was no statistically significant difference between the groups. The total IgE level in blood was elevated after the test was completed (day 43), but the collagen administration group had lower total IgE levels in blood than the control feed group, although there was no statistically significant difference.

### (4) TEWL Measurement

FIG. 5 shows the TEWL changes in each group. The data taken before the start of feeding (day 0), on day 15 and day 29, and after the completion of the feeding period (day 43) are shown as the average value±standard error (7 mice per group).

TEWLs in both groups at the time of starting feeding were 5 g/hr/m² or less, which were within the normal range. From day 15 after the start of feeding or later, TEWL showed an increasing tendency, particularly increased over time in the control feed administration group, reaching a rate as high as 25.97±3.85 g/hr/m² at the time of completing the feeding period (day 43). TEWL also increased in the collagen administration group but was as slightly low as 23.72±9.38 g/hr/m² at the time of completing the feeding period, although there was no significant difference.

### (5) Measurement of Body Weight

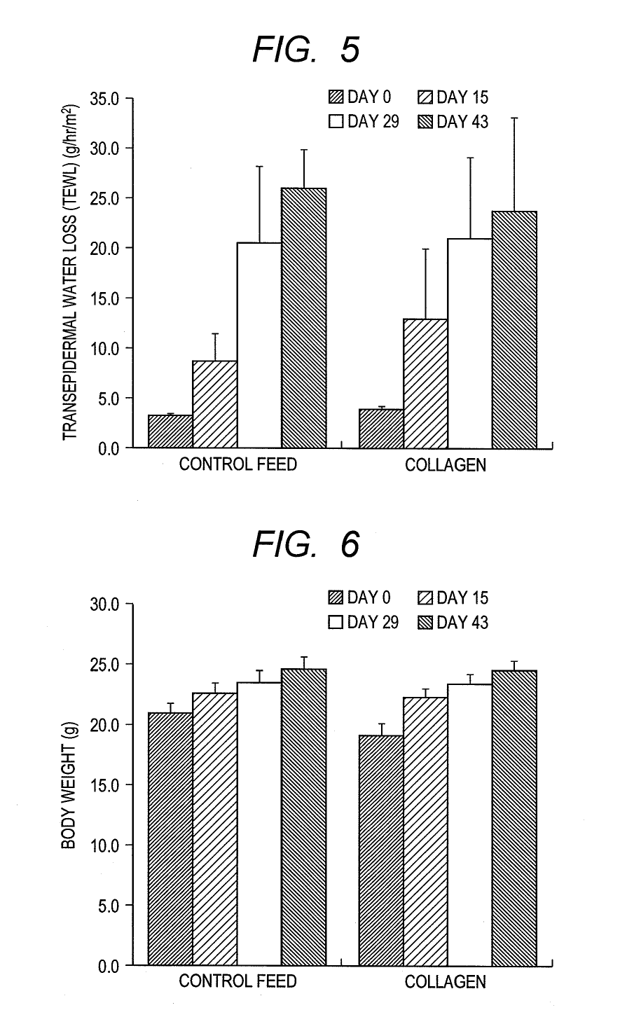

FIG. 6 shows the changes in body weight in each group. The data taken before the start of feeding (day 0), on day 15 and day 29, and after completing the feeding period (day 43) are shown as the average value±standard error (7 mice per group).

The body weights at the time of starting feeding were 18.7 to 20.8 g. Then, the body weights increased over time in both groups with no difference in the increase rate between the groups.

### (6) Macroscopical Observation of Skin Lesion

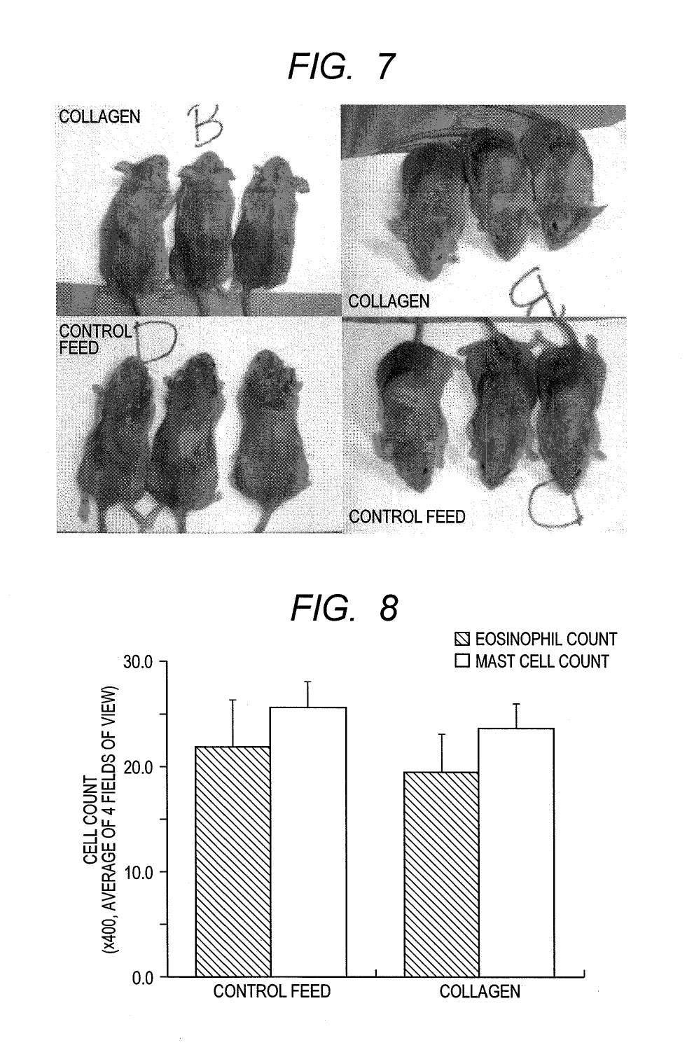

FIG. 7 shows the macroscopic findings in each group. These macrophotogrpahs were taken before collecting samples for (7) histological examination from the mice of each group after the completion of the feeding period.

B; collagen, D; control feed. When the macroscopic findings of the mouse head dorsal part and face of each group at the time of test completion were compared, the control feed administration group was found to have progressed dermatitis at each site. In the collagen administration group, on the other hand, the dermatitis occurred, but was only mild.

### (7) Histological Examination

FIG. 8 shows the eosinophil counts and mast cell counts in the head dorsal skin tissues in each group. These counts are shown as the average value±standard error (7 mice per group) of the results of counting the skin tissues collected after the completion of the feeding period (day 43). The collagen administration group, compared with the control feed group, had less cell numbers in both eosinophil count and mast cell count, although there was no significant difference.

### Conclusion of Example 1

No dermatitis was found in any of the tests at the time of starting feeding for both groups.

Both groups developed the dermatitis since then; however, the collagen-containing feed administered NC/Nga Tnd mice, when compared with the control group, had decreased values in the clinical symptom score, scratching frequency and scratching duration, total IgE level in blood, TEWL, macroscopical observation of the lesions and histological examination. Based on these findings, it is verified that the collagen administration before the onset is effective to prevent atopic dermatitis.

Also, no difference in the body weight increase was observed in the collagen administration group from the control feed administration group, which confirmed the safety of the collagen administration.

In accordance with the following formulae, drinks, a powder, a tablet, a chewing gum, a candy and a tablet candy were produced.

### Example 2

### Formula of Drink

Collagen peptide 5.0 parts by weight

High fructose corn syrup 8.0 parts by weight

Sugar 4.0 parts by weight

Flavor 0.5 parts by weight

Vitamin C 5.0 parts by weight

After adjusting pH to 3.8 using an acidifier, purified water was added to make 100 parts by volume.

### Example 3

### Formula of Drink

Collagen peptide 5.0 parts by weight

Sucralose 0.005 parts by weight

Stevioside 0.008 parts by weight

Rebaudioside 0.008 parts by weight

Acesulfame potassium 0.01 parts by weight

Peach flavor 0.5 parts by weight

Vitamin C 0.5 parts by weight

After adjusting pH to 3.8 using an acidifier, purified water was added to make 100 parts by volume.

### Example 4

### Formula of Drink

Collagen peptide 5.0 parts by weight

Lactic beverage 5.0 parts by weight

High fructose corn syrup 10.0 parts by weight

Flavor 0.5 parts by weight

Vitamin C 5.0 parts by weight

After adjusting pH to 3.8 using an acidifier, purified water was added to make 100 parts by volume.

### Example 5

### Formula of Drink

Collagen peptide 5.0 parts by weight

High fructose corn syrup 10.0 parts by weight

Honey 5.0 parts by weight

Flavor 0.5 parts by weight

Vitamin C 5.0 parts by weight

After adjusting pH to 3.8 using an acidifier, purified water was added to make 100 parts by volume.

### Example 6

### Formula of Jelly Drink

Collagen peptide 5.0 parts by weight

Sucralose 0.005 parts by weight

Stevioside 0.008 parts by weight

Rebaudioside 0.008 parts by weight

Acesulfame potassium 0.01 parts by weight

Peach flavor 0.5 parts by weight

Vitamin C 5.0 parts by weight

Stabilizer for gelation 0.5 parts by weight

After adjusting pH to 3.8 using an acidifier, purified water was added to make 100 parts by volume.

### Example 7

### Formula of Jelly Drink

Collagen peptide 5.0 parts by weight

High fructose corn syrup 8.0 parts by weight

Sugar 4.0 parts by weight

Flavor 0.5 parts by weight

Vitamin C 5.0 parts by weight

Stabilizer for gelation 0.5 parts by weight

After adjusting pH to 3.8 using an acidifier, purified water was added to make 100 parts by volume.

### Example 8

### Formula of Coffee Drink

Collagen peptide 5.0 parts by weight

Coffee extracts 5.0 parts by weight

Sugar 4.0 parts by weight

Flavor 0.5 parts by weight

Vitamin C 0.5 parts by weight

After adjusting pH to 6.5 using sodium bicarbonate, purified water was added to make 100 parts by volume.

### Example 9

### Formula of Green Tea Drink

Collagen peptide 5.0 parts by weight

Green tea extracts 10.0 parts by weight

Flavor 0.5 parts by weight

Vitamin C 0.5 parts by weight

After adjusting pH to 6.5 using sodium bicarbonate, purified water was added to make 100 parts by volume.

### Example 10

### Formula of Powder

Collagen peptide 90.0 parts by weight

Lactose 5.0 parts by weight

Dextrin 4.0 parts by weight

Vitamin C 1.0 parts by weight

### Example 11

### Formula of Tablet

Collagen peptide 5.0 parts by weight

D-mannitol 40.0 parts by weight

Lactose 40.0 parts by weight

Crystalline cellulose 10.0 parts by weight

Hydroxypropyl cellulose 5.0 parts by weight

### Example 12

### Formula of Chewing Gum

Collagen peptide 5.0 parts by weight

Gum base 20.0 parts by weight

Sugar 55.0 parts by weight

Glucose 10.5 parts by weight

Starch syrup 9.0 parts by weight

Flavor 0.5 parts by weight

### Example 13

### Formula of Candy

Collagen peptide 5.0 parts by weight

Sugar 50.0 parts by weight

Starch syrup 29.5 parts by weight

Flavor 0.5 parts by weight

Water 15.0 parts by weight

### Example 14

### Formula of Tablet Candy

Collagen peptide 5.0 parts by weight

Sugar 73.5 parts by weight

Glucose 17.0 parts by weight

Sucrose esters of fatty acids 0.2 parts by weight

Flavor 0.2 parts by weight

Water 4.1 parts by weight

### Example 15

### Formula of Gummy Jelly

Collagen peptide 5.0 parts by weight

Gelatin 55.0 parts by weight

Starch syrup 23.0 parts by weight

Sugar 8.5 parts by weight

Vegetable oil 4.5 parts by weight

Mannitol 3.0 parts by weight

Lemon juice 1.0 parts by weight

### Example 16

### Formula of Chocolate

Collagen peptide 5.0 parts by weight

Powdered sugar 36.8 parts by weight

Cacao bitter 20.0 parts by weight

Whole milk powder 20.0 parts by weight

Cacao butter 17.0 parts by weight

Mannitol 1.0 parts by weight

Flavor 0.2 parts by weight

### Example 17

### Formula of Sorbet

Collagen peptide 5.0 parts by weight

Orange juice 25.0 parts by weight

Sugar 23.0 parts by weight

Albumen 9.0 parts by weight

Water 38.0 parts by weight

This application claims the priority to the Japanese Patent Application No. 2009-280606, filed on Dec. 10, 2009, and the disclosure of which is hereby incorporated by reference as a part of the present application.

1. A preventive agent for atopic dermatitis comprising collagen.

2. A food and beverage product containing a preventive agent according to claim 1.
